(19) United States
(12) Patent Application Publication
(10) Pub. No.: US 2008/0218696 A1
(43) Pub. Date: Sep. 11, 2008

(54) Non-Invasive Monitoring System

(76) Inventor: Jose Mir, Rochester, NY (US)

Correspondence Address: JAECKLE FLEISCHMANN & MUGEL, LLP, 190 Linden Oaks, ROCHESTER, NY, 14625-2812, US

(21) Appl. No.: 11/994,444

(22) PCT Filed: Jun. 30, 2006
(86) PCT No.: PCT/US06/26105

§ 371 Date: Jan. 2, 2008

Related U.S. Application Data
(60) Provisional application No. 60/696,311, filed on Jul. 1, 2005.

(51) Int. Cl. A61B 3/10; G02C 7/04

(52) U.S. Cl. 351/219; 351/177

(57) ABSTRACT

A monitoring system includes a light source that illuminates at least a portion of a subject's eye with an incident light beam, and a contact lens with a coupler. The coupler couples the incident light beam into an aqueous humor of the eye, creating an aqueous light beam. The coupler also couples the aqueous light beam out of the aqueous humor of the eye, creating an output light beam. The monitoring system also includes a sensor that measures at least one spectral characteristic of the output light beam. The monitoring system further includes a processing system that determines at least one measurable characteristic of the subject based on the at least one spectral characteristic of the output light beam. A method for monitoring is provided, as well as a contact lens for use with a monitoring systems, and a method of manufacturing a contact lens.

### RELATED APPLICATIONS

This application claims priority to U.S. provisional application number 60,696,311, filed on Jul. 1, 2005, entitled, “Non-invasive, Spectral, Glucose Monitoring Systems and Methods Thereof,” the entire specification of which is hereby officially incorporated by reference.

### TECHNICAL FIELD

The claimed invention relates to monitoring systems, more particularly to a non-invasive spectral monitoring system for measuring characteristics of a subject; components of such a system; and methods thereof.

### BACKGROUND

It is estimated that diabetes affects 5-10% of the population. Periodic glucose monitoring is critical to diabetic patients since blood sugar can change rapidly to dangerous levels. Unfortunately, most glucose monitors are invasive and require blood samples obtained with fingersticks and other painful, inconvenient methods. As a result, attempts have been made to develop non-invasive monitors using optical techniques.

Non-invasive methods to monitor glucose exist that rely on the dependence of refractive index, optical activity, or absorption spectra of the aqueous humor of a subject's eye versus glucose concentration. Since the chemical composition of the aqueous humor is representative of blood chemistry, these approaches attempt to provide non-invasive techniques to monitor blood glucose levels.

Unfortunately, there are several practical problems that make these types of measurements difficult and inconvenient to make. For example, referring to FIG. 1A the aqueous humor 20 of a subject's eye 22 may be monitored using an input light beam 24 that traverses the aqueous humor 20 and then is reflected by the aqueous humor 20/lens 26 interface. Although there is a measurable refractive index difference between the aqueous humor 20 and the eye lens 26, the difference is small and only a small percentage of the light is reflected. This low reflectance results in feeble signals and low signal-to-noise ratios.

Another problem with reflecting light off of the aqueous humor 20/lens 26 interface is the short optical path through the aqueous humor 20, since infrared absorption, polarimetry, and refractometry depend on both material property changes and optical path length. This problem is of greatest concern for measurements made along the eye's optical axis 28, since the optical path length is minimized in this geometry.

Referring to FIG. 1B, attempts have been made to refract light beams 30 from the side of the eye 22 to increase optical path length and avoid low reflectivity at the aqueous humor 20/lens 26 interface. Unfortunately, because of cornea 31 geometry and mean refractive index (approximately 1.33), oblique angles are required as shown in FIG. 1B.

Referring more specifically to FIG. 1C, an incident light beam 30 makes an angle 32 from a surface normal 34. The light beam 30 is refracted at an angle 36 from the surface normal 34. In the orientation shown, the refracted light beam 38 must propagate in a horizontal direction to maximize optical path length and provide a way to output the light beam at the other side of the eye. As a result, much light is lost when refracted light beam 38 reaches the other side of the eye (not shown) due to internal reflection at this surface. Furthermore, using the method of FIGS. 1B and 1C, the light beam 30 must be input and the refracted beam 38 output at angles that are highly sensitive to eye position, inconvenient for the measurement system, and inconvenient for the human or animal subject. Such inconvenient angles also increase the size, reliability, and subsequently the cost of systems with this architecture.

Therefore, there exists a need for an easy-to-use, convenient, reliable, and low-cost non-invasive monitoring system for measuring subject characteristics.

### SUMMARY

A monitoring system includes a light source that illuminates at least a portion of a subject's eye with an incident light beam, and a contact lens with a coupler. The coupler couples the incident light beam into an aqueous humor of the eye, creating an aqueous light beam. The coupler also couples the aqueous light beam out of the aqueous humor of the eye, creating an output light beam. The monitoring system also includes a sensor that measures at least one spectral characteristic of the output light beam. The monitoring system further includes a processing system that determines at least one measurable characteristic of the subject based on the at least one spectral characteristic of the output light beam.

A method for monitoring is provided. At least a portion of a subject's eye is illuminated with a light beam. The light beam is coupled into an aqueous humor of the eye with a coupler contact lens. The light beam coupled into the aqueous humor with the coupler contact lens is output. At least one spectral characteristic of the output light beam is measured. One or more measurable characteristics of the subject are calculated based on the at least one measured spectral characteristic.

A body-worn monitoring system includes an article which can be worn by a subject, a light source coupled to the article that illuminates at least a portion of the subject's eye with an incident light beam, and a contact lens with a coupler. The coupler couples the incident light beam into an aqueous humor of the eye, creating an aqueous light beam. The coupler also couples the aqueous light beam out of the aqueous humor of the eye, creating an output light beam. The body-worn monitoring system also includes a sensor coupled to the article that measures at least one spectral characteristic of the output light beam. The body-worn monitoring system further includes a processing system coupled to the sensor that calculates at least one measurable characteristic of the subject.

A contact lens includes a first coupler for directing incident light through an aqueous humor, and a second coupler for receiving light directed from the first coupler and directing that light out of the aqueous humor and away from the contact lens.

A method of manufacturing a contact lens is provided. A lens substrate is formed. A coupler is formed on the lens substrate, such that the coupler can direct incident light behind the contact lens, through a medium the contact lens will be worn on, and back out of the contact lens.

The claimed invention provides a convenient way to optically monitor a subject's characteristics, such as glucose level, using an optoelectronic system that senses spectral content of light sampling the aqueous humor. Due to its compact size, its possibility for fully integrated functions, and simple alignment requirement, this technique can be made portable and could be incorporated into personal eyewear. The portability and ease-of-use advantages of this method provide a unique approach to do continuous glucose analysis as required by the individual's health needs. A further advantage of the claimed invention is that it can optimize the optical path length within the aqueous humor, thereby increasing sensitivity and signal-to-noise ratio. A further advantage of the claimed invention is that it provides a convenient geometry for inputting and outputting the probing light, enhancing its usability. A further advantage of the claimed invention is that it provides a method for non-invasive measurement which is substantially less sensitive to eye position and motion.

### BRIEF DESCRIPTION OF THE DRAWINGS

FIG. 1A is a cross-sectional view of an eye with an incident light beam striking a portion of the eye from a direction close to the optical axis of the eye.

FIG. 1B is a cross-sectional view of an eye with an incident light beam striking a portion of the eye from an oblique angle.

FIG. 1C is an enlarged view of a portion of the incident light beam striking the eye from FIG. 1B.

FIG. 2A schematically illustrates one embodiment of a monitoring system.

FIG. 2B is an enlarged view of a portion of the embodied monitoring system of FIG. 2A.

FIGS. 3A-3B schematically illustrate different embodiments of body-worn monitoring systems.

FIGS. 4-7 schematically illustrate different embodiments of monitoring systems.

FIGS. 8A-8D schematically illustrate different embodiments of contact lenses for use as part of a monitoring system.

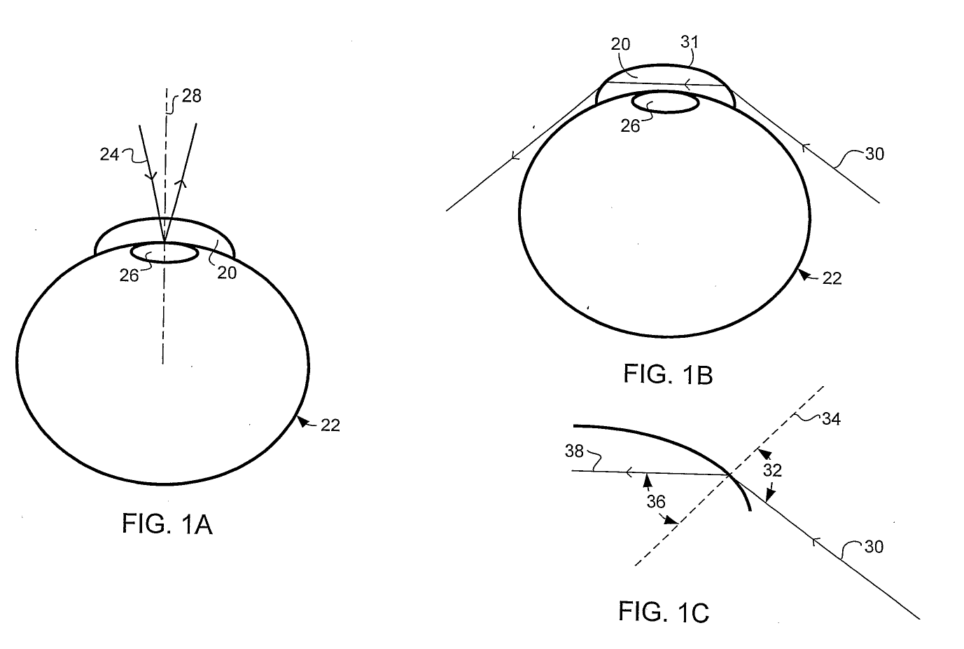

### DETAILED DESCRIPTION

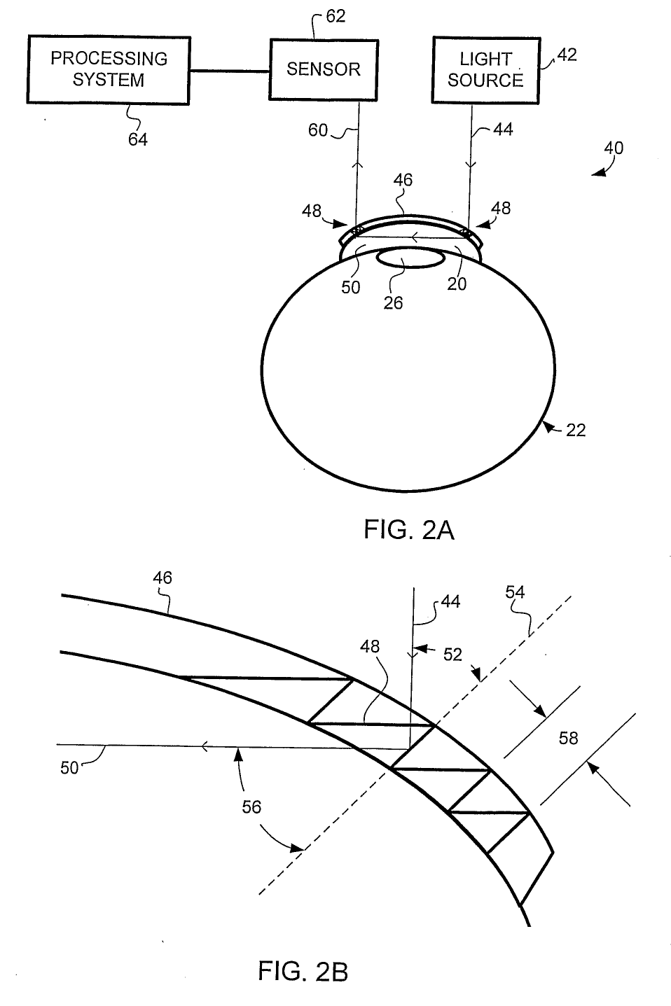

FIG. 2A schematically illustrates one embodiment of a monitoring system 40. The monitoring system 40 may be used to determine at least one measurable characteristic of a subject. The subject could be any person or animal having an eye with an aqueous humor 20 or similar fluid-filled space. For simplicity, only the eye 22 of the subject is illustrated. The eye is shown schematically, illustrating relevant portions of the eye to facilitate explanations. The measurable characteristic determined by the monitoring system 40 may include chemical or physical characteristic of the subjects blood, since the fluid of the aqueous humor 20 is known to be representative of blood plasma. Examples of physical or chemical characteristics of the subject's blood may be, but are not limited to, blood pressure, glucose concentration, blood alcohol level, cholesterol level, HDL cholesterol, estrogen, progesterone, and cortisol. Other measurable characteristics determined by the monitoring system 40 may include ocular characteristics, such as, but not limited to the health of the aqueous humor 20.

The monitoring system 40 has a light source 42 that illuminates at least a portion of the subject's eye 22 with an incident light beam 44. The term “light beam” as it is used in this specification is intended to include, but not be limited to light from a point source, columnar light, imaged and/or focused light, optically directed light, and filtered light. Depending on the embodiment, many different light sources could be used for light source 42. Examples include light bulbs, light emitting diodes (LED's), fiber optic light sources, multi-wavelength LED arrays, solid state lasers, and even combinations thereof. The choice of light source 42 in a given embodiment can be influenced by the subject characteristic being monitored. Light source 42 should be selected so that at least a portion of its emission wavelength(s) overlap with the absorption wavelength(s) of the chemical or characteristic being measured.

The monitoring system in the embodiment of FIG. 2A also has a contact lens 46 with a coupler 48 that couples the incident light beam 44 into the aqueous humor 20 of the eye 22, creating an aqueous light beam 50. The aqueous light beam 50 is defined as the light beam that is coupled and propagates through the aqueous humor 20. FIG. 2B is an enlarged view of a portion of the incident light beam 44 striking the eye 22 from FIG. 2A. The incident light beam 44 forms an angle 52 with a surface normal 54. The contact lens 46 with an integral coupler 48 couples the incident light beam 44 at a directed angle 56 away from the surface normal 54, forming the aqueous light beam 50. Coupler 48 is illustrated schematically, but may comprise reflective, diffractive, and/or refractive serrated elements of spacing 58 that cause the coupled aqueous light beam 50 to traverse the aqueous humor 20.

Approximate conditions for the dimensions of spacing 58 depend largely on the contact lens 46, itself. Since the geometry of the pattern allows a large amount of the incident light 44 to be coupled into the aqueous humor 20, its spacing 58 should be substantially continuous, lacking significant gaps, although gaps between serrated elements of the coupler 48 may be possible in other embodiments. In this embodiment, the serrated pattern has a low profile relative to the contact lens 46 surface in order to help provide a comfortable wear to the user. The spacing 58 can vary broadly from tenths of microns (blazed gratings) to approaching the entire thickness of the contact lens 46, on the order of one millimeter. In some embodiments, the spacing 58 will be approximately in the 20-200 micron range where optical coupling characteristics such as efficiency and angular spread will have lower wavelength dependence while comfort for the subject will be acceptable.

Referring again the monitoring system 40 embodiment illustrated in FIG. 2A, the coupler 48 has characteristics, such as the ones described above, to propagate the incident light beam 44 through the aqueous humor 20 in the form of an aqueous light beam 50. The coupler 48 then couples the propagating aqueous light beam 50 out of the aqueous humor 20, creating an output light beam 60. If the output light beam 50 side of the coupler 48 is similar to the incident light beam 44 side of the coupler 48, then the output light beam 50 will be coupled out of the aqueous humor 20 at an angle which is approximately 180 degrees from the original incident angle.

The monitoring system 40 also has a sensor 62 that measures at least one spectral characteristic of the output light beam 60. An example of a spectral characteristic may include absorption spectra. Examples of a sensor 62 which would be compatible with the monitoring system include, but are not limited to a spectrometer, a microspectrometer, and a photo-sensitive application specific integrated circuit (ASIC). The sensor 62 preferably has sensitivity in the spectral region of interest which correlates to the characteristic being monitored.

A processing system 64 is functionally coupled to the sensor 62. The processing system 64 determines at least one measurable characteristic of the subject, based on the data collected by the sensor 62. For example, if the measurable characteristic of interest was glucose concentration, the processing system 64 could be calculated from the measured absorption spectrum by sensor 62. Near infrared spectral ranges of 400-4000 cm-1 and 4000-10000 cm-1 may be used for glucose, ethanol, and urea since they exhibit absorption bands in these ranges, although other types of ranges and sensors 62 may be used. Since other species, such as water, exhibit strong absorption bands in the same spectral region as glucose, their contribution needs to be subtracted out. This may be done by using multivariate spectral analysis or by subtracting the corresponding water spectral absorption from a calibrated water sample of known optical path length. For calibration readings taken on the subject prior to test conditions, the delay between glucose levels measured at the aqueous humor relative to blood glucose measurements should be taken into account. This delay can be on the order of ten to twenty minutes for glucose concentration, and may be more or less for other characteristics being measured. Calibrations should account for these delays for best accuracy.

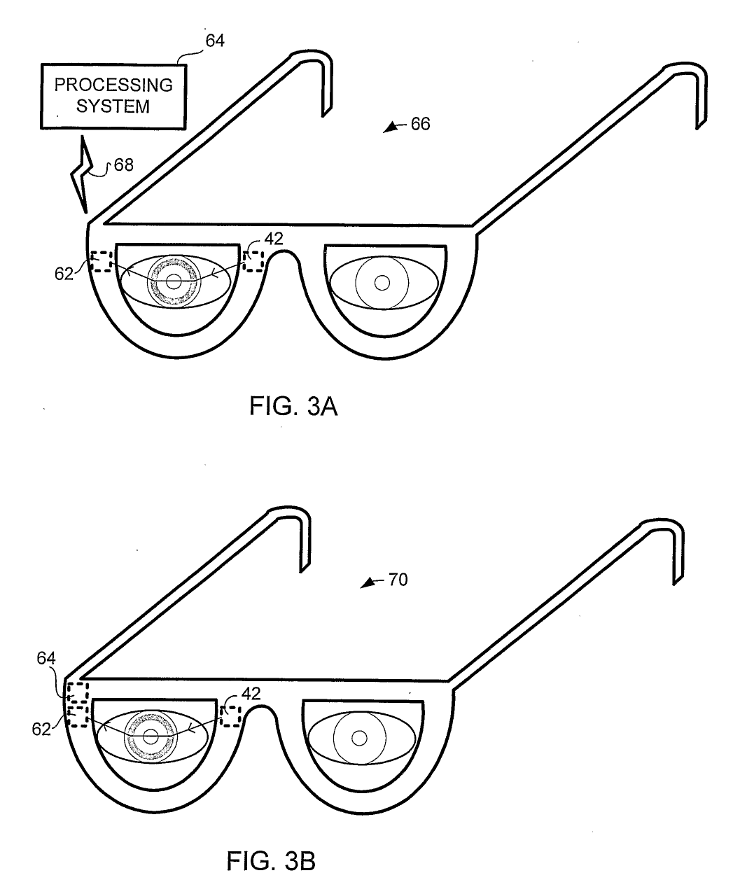

The processing system 64 can have a central processing unit (CPU) or processor and a memory which are coupled together by a bus or other link. Alternatively, processing system 64 could include a computer, an application specific integrated circuit (ASIC), digital components, analog components, wireless and/or hardwired communications links, a microprocessor, volatile and/or non-volatile memory, hard drives, disk drives, other storage devices, or any combination thereof. The processing system 64 may be distributed, such that at least one portion of the processing happens in a substantially different location. For example, the monitoring system 40 could be a body worn monitoring system 66, such as the embodiment illustrated in FIG. 3A, where the processing system 64 is wirelessly coupled 68 to the sensor 62. The wireless coupling 68 could be any one-way or two-way wireless communication protocol, such as, for example, Bluetooth, IEEE 802.11, or an encrypted protocol. In the embodiment of FIG. 3A, the light source 42 and sensor 62 are integral to a pair of eyeglasses, while the processing system 64 is located remotely to the user. Other body-worn monitoring systems 66 could be built into other personal eyewear, such as sunglasses, visors, goggles, and masks, as well as into hats, clothing, and helmets. FIG. 3B schematically illustrates another embodiment of a monitoring system 40, here illustrated as a body-worn monitoring system 70. Body-worn monitoring system 70 is similar to the monitoring system of FIG. 3A, with the exception that the processing system 64 is directly coupled to the sensor 62, rather than remotely coupled as in FIG. 3A. In any of the embodiments, the processing system 64 may also include a user interface component which can give detailed reading information on a computer screen or LCD panel, sound alerts, vibrate, and/or turn indicator lights or LED's on and off.

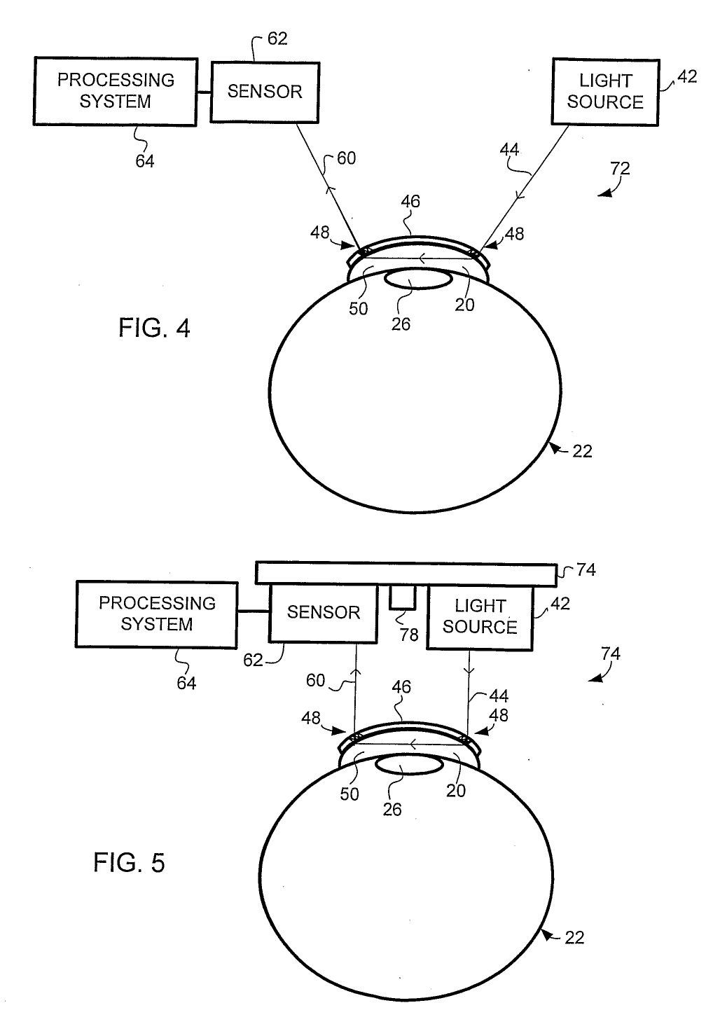

The embodiments illustrated in FIGS. 3A and 3B bring up a feature that the coupler 48 of the contact lens 46 may be designed to have, such that the incident light beam 44 and the output light beam 60 do not have to be substantially parallel to each other. In some embodiments, such as the one of FIG. 2A, it may be preferable to have the output light beam 60 exit 180 degrees from the incident light beam, especially if the light source 42 and the sensor 62 are located close to each other, for example, on a common substrate or circuit board. In other embodiments, however, such as the ones in FIGS. 3A and 3B, it may be preferable to have the output light beam 60 exit at an angle which is not parallel to the incident light beam 44, while still using the coupler 48. An example of a such an embodiment is illustrated by the monitoring system 72 in FIG. 4. In this case, the coupler optical characteristics would need to be selected so as to result in the desired input and output angles. While FIG. 4 illustrates substantially symmetrical incident and output light beams, these light beams do not have to be symmetrical.

FIG. 5 illustrates another embodiment of a monitoring system 74, wherein the light source 42 and the sensor 62 are mounted on or coupled to a substrate 76. In this embodiment, the substrate 76 serves to provide a constant spatial relationship for sensor 62 relative to light source 42. A proper alignment of the contact lens 46 and its coupler 48 may also be needed to properly complete the optical path between the light source 42 and the sensor 62. One way to accomplish this alignment is to provide a reference alignment optical marker 78 coupled to the substrate 76 whereby the subject can be instructed to look at the alignment marker 78 as the measurement is made. The instruction to look at the alignment marker 78 can be manually given in some embodiments. In other embodiments, the processing system 64 may have a user interface which capable of automatically instructing the subject to look at the alignment mark 78 using, for example, a sound alert, a vibration device, an indicator light, and/or a recorded message. An additional instruction could be given or indicated to inform the subject of the completion of the measurement process.

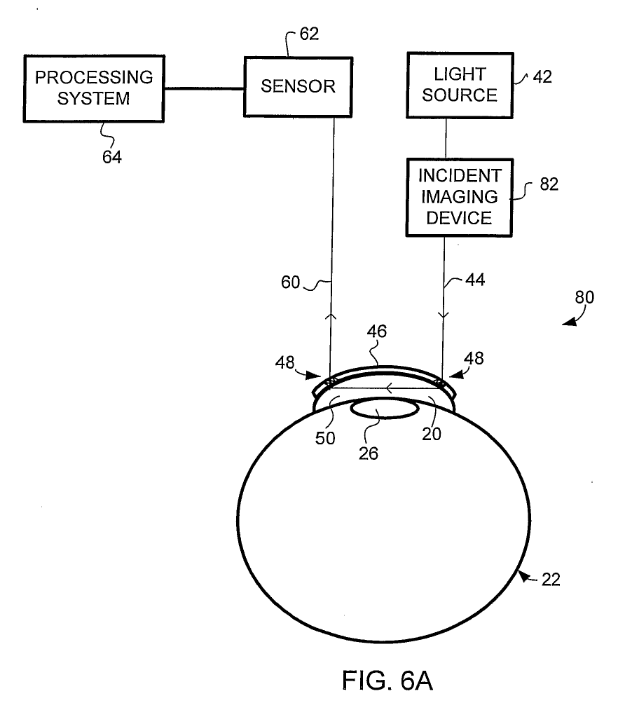

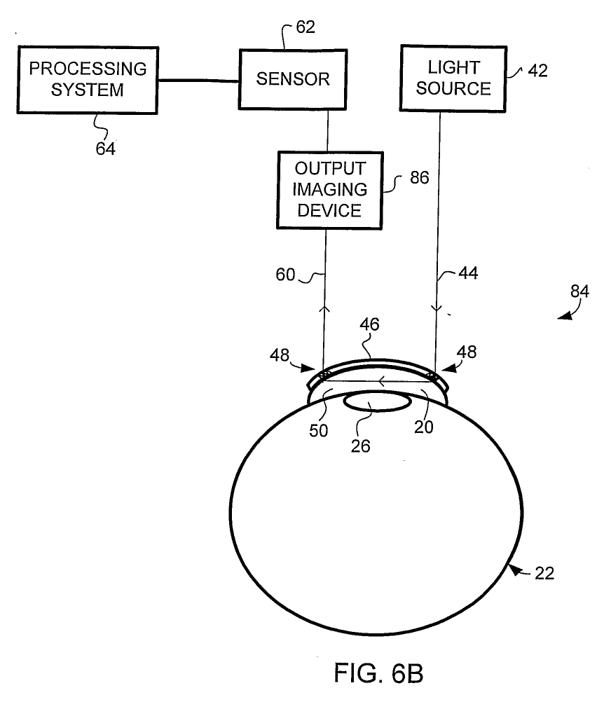

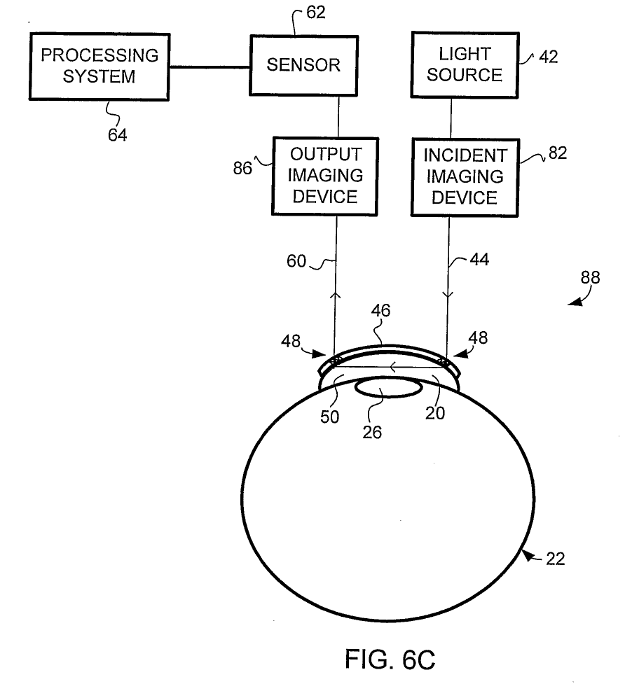

FIGS. 6A-6C schematically illustrate further embodiments of a non-invasive monitoring system. In the monitoring system 80 embodied in FIG. 6A, an incident imaging device 82 is provided between the light source 42 and the coupler 48 to focus the incident light beam 44 on at least a portion of the coupler 48. In the monitoring system 84 embodied in FIG. 6B, an output imaging device 86 is provided between the coupler 48 and the sensor 62 to focus the output light beam 60 on the sensor 62. In the embodied monitoring system 88 of FIG. 6C, both an incident imaging device 82 and an output imaging device 86 are provided. Although some embodiments may wish not to use either an incident imaging system 82 and/or an output imaging system 86, many will wish to have these imaging devices to increase the signal-to-noise and efficiency of the monitoring system.

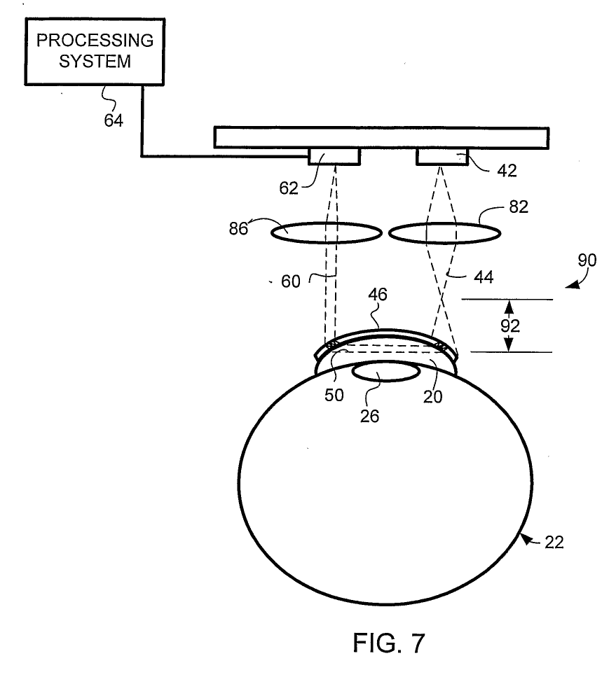

The monitoring system embodied in FIG. 7 schematically illustrates a more specific example of how a monitoring system 90 might look with both an incident imaging system 82 and an output imaging system 86, here illustrated as double convex lenses. It should be understood that other imaging systems 82, 86 could have other types of lenses and/or combinations of lenses. Single lenses are illustrated in the embodiment of FIG. 7 for simplicity. In this embodiment, the incident imaging system 82 produces a focused spot at a specific distance 92 from the eye 22. Distance 92 is chosen to approximately equal the effective focal length of the contact lens 46 combined with the cornea. A typical value for distance 92 is 11 mm, but this distance can vary depending on the subject and other conditions.

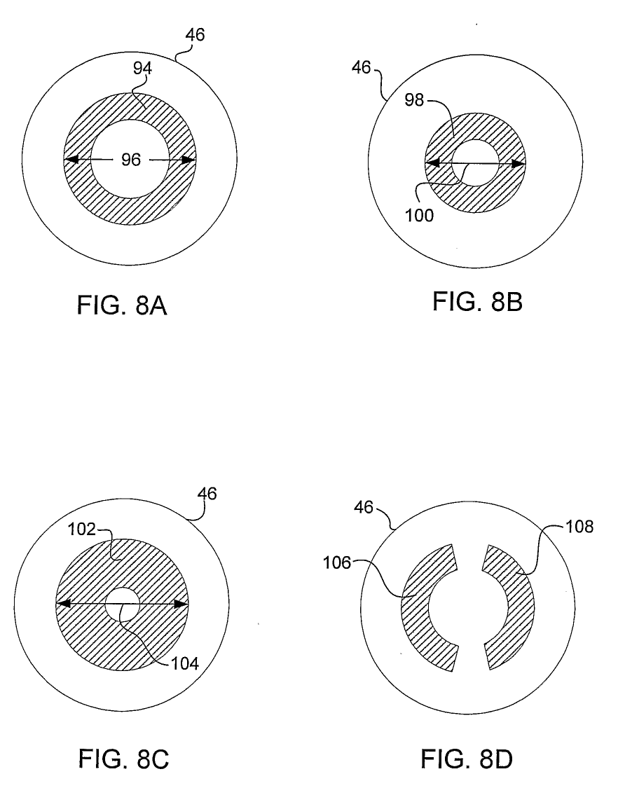

FIGS. 8A-8C schematically illustrate embodiments of contact lenses suitable for use in the monitoring systems described herein and their equivalents. The geometry of the coupler on the contact lens will determine the effective path length, the cornea optic power to be compensated for by distance 92 (from FIG. 7) and available area for light beam to propagate. For convenience, the coupler regions of the contact lenses in the drawings are shaded in order to clearly identify where the regions are in relation to the contact lens. In real practice, the coupler regions may or may not be visible to the unaided eye. FIG. 8A illustrates an embodiment of a contact lens 46 with a coupler 94 having a maximum optical path length of distance 96. FIG. 8B illustrates another embodiment of a contact lens 46 with a coupler 98 having a maximum optical path length of distance 100, which is shorter than optical path length 96 due to the smaller diameter of the coupler 98. FIG. 8C illustrates a further embodiment of a contact lens 46 with a coupler 102 having a maximum optical path length of distance 104, which is substantially equal in length to the optical path length of the lens in FIG. 8A. The lens in FIG. 8C, however, has a larger area for the light beam to propagate, since the coupler 102 is larger in area than the coupler 94 of FIG. 8A.

In the embodiments of FIGS. 8A-8C, each coupler 94, 98, 102 comprises a ring-shaped coupler. The diffusive, refractive, reflective, and/or diffractive elements within the couplers may also be radially symmetric around the contact lens. The advantage of this geometry in a ring-shaped coupler is that the measurement is independent of the rotation of the contact lens 46 relative to the eye of the subject. Ideally, the ring-shaped pattern should be wide enough to provide sufficient signal at the sensor 62, but not too wide as to direct (through diffraction, reflection, refraction, and/or diffusion) unwanted light into the pupil.

Other embodiments of contact lenses for use in the monitoring system may have geometries which are not ring-shaped. For example, in the embodiment of FIG. 8D, the coupler is separated into a first coupling element 106 and a second coupling element 108 which are not continuous with each other. The first coupling element 106 and the second coupling element 108 can have the same or different optical elements for diffraction, diffusion, reflection, and/or refraction, as can different areas of the continuous ring-shaped embodiments. When there are differences in optical elements on the same coupler (whether continuous or not) the contact lens will have to be aligned properly with the light source and the sensor of the monitoring system. This alignment could be done manually or through a recognition and adjustment device coupled to the monitoring system.

The couplers in the monitoring systems described herein, and their equivalents may include reflective, diffractive, diffusive, and/or refractive elements. Reflective optical elements have already been discussed with regard to FIG. 2B above. Couplers with diffractive optical elements may involve one or more gratings that satisfy the condition:

$$sin ( θ ) = [ \frac{λ m}{nd} ] - \frac{sin ( ϕ )}{n}$$

where, referring to FIG. 2B, φ is the magnitude of the incident angle 52, θ is the magnitude of directed angle 56, m is the diffractive order, d is the grating spacing 58, n is the refractive index of the aqueous humor 22, and λ is the wavelength of the incoming light beam. For example, using values of λ=0.83 microns, d=0.5 microns, n=1.33, and φ=45 degrees, results in a diffracted first order approximately propagating so as to maximize the optical path length along the aqueous humor. (i.e. a substantially horizontal aqueous light beam 50 propagating from right to left in the orientation of FIG. 2A). If several wavelengths need to be monitored, a set of gratings with spacings that satisfy the above equation may be used. The diffraction gratings may be blazed in order to maximize the amount of light diffracted into the desired order.

Other embodiments of couplers may wish to use diffusive elements. In this case, light is coupled in a broad range of angles within the aqueous humor. Due to their angular dispersion, diffusive couplers provide a lower degree of efficiency and control than other methods.

Throughout this specification, the contact lens is referred to as having a coupler. The coupler can be thought of as having a first coupler for coupling the incident light beam into the aqueous humor, and as having a second coupler for coupling the aqueous light beam out of the eye. These first and second couplers may be continuous as is FIGS. 8A-8C, or discontinuous as in FIG. 8D. The first and second couplers may have similar or different optical elements. The first and second couplers may also simply be referred to as “the coupler” on the contact lens, since it is understood that the coupler has first and second or input and output components.

The contact lens embodiments discussed in this specification, and their equivalents may be manufactured from a variety of methods. The lens substrate may be formed of plastic, polymer, glass, or similar suitable material. The lens may optionally be formed with a vision correction element in the portion of the lens which will go over the pupil. Different sized lenses are contemplated for varying eyeball shapes. In another manufacturing action, a coupler is formed on the lens substrate such that the coupler can direct incident light behind the contact lens, through a medium the contact lens will be worn on, and back out of the contact lens. The formation of the coupler can be accomplished by embossing the lens substrate with an embossing mold. The embossing mold may have a diffraction grating pattern, a diffusive pattern, a reflection pattern, or any combination thereof.

The formation of the coupler may alternatively or additionally be accomplished by combining two materials with different refractive indexes to form a serrated pattern.

The formation of the coupler may alternatively or additionally be accomplished by adding reflective material at the serrated surface.

The advantages of a non-invasive monitoring system have been discussed herein. Embodiments of a non-invasive monitoring system, including a contact lens with a coupler for use in the monitoring system, as well as methods for using this system have been described by way of example in this specification. It will be apparent to those skilled in the art that the forgoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A monitoring system, comprising:
a light source that illuminates at least a portion of a subject's eye with an incident light beam;
a contact lens with a coupler that:
couples the incident light beam into an aqueous humor of the eye, creating an aqueous light beam; and
couples the aqueous light beam out of the aqueous humor of the eye, creating an output light beam;
a sensor that measures at least one spectral characteristic of the output light beam; and
a processing system that determines at least one measurable characteristic of the subject based on the at least one spectral characteristic of the output light beam.

2. The monitoring system of claim 1, wherein the coupler comprises:
a first serrated optical coupler that couples the incident light beam into the aqueous humor of the eye; and
a second serrated optical coupler that couples the aqueous light beam out of the aqueous humor of the eye.

3. The monitoring system of claim 2, wherein the geometry of the first serrated optical coupler is substantially the same as the geometry of the second serrated optical coupler.

4. The monitoring system of claim 2, wherein the first serrated optical coupler is continuous with the second serrated optical coupler.

5. The monitoring system of claim 1, wherein the coupler comprises a ring-shaped serrated optical coupler.

6. The monitoring system of claim 1, wherein the coupler comprises:
a first set of diffractive gratings on a contact lens that couples the incident light beam into the aqueous humor; and
a second set of diffractive gratings that couples the aqueous light beam out of the aqueous humor.

7. The monitoring system of claim 6, wherein:
the first set of diffraction gratings have first diffraction elements;
the second set of diffraction gratings have second diffraction elements; and
the spacing of the first diffraction elements and the spacing of the second diffraction elements are substantially the same.

8. The monitoring system of claim 6, wherein the first set of diffraction gratings are continuous with the second set of diffraction gratings.

9. The monitoring system of claim 6, wherein the first set of diffraction gratings and the second set of diffraction gratings comprise a ring-shaped diffraction grating on the contact lens.

10. The monitoring system of claim 1, wherein the coupler comprises:
a first diffusive element on the contact lens that couples the incident light beam into the aqueous humor; and
a second diffusive element on the contact lens that couples the aqueous light beam out of the aqueous humor.

11. The monitoring system of claim 10, wherein:
the first diffusive element has first optical properties;
the second diffusive element has second optical properties; and
the first optical properties and the second optical properties are substantially the same.

12. The monitoring system of claim 10, wherein the first diffusive element on the contact lens is continuous with the second diffusive element.

13. The monitoring system of claim 10, wherein the first diffusive element and the second diffusive element comprise a ring-shaped diffusive element on the contact lens.

14. The monitoring system of claim 1, further comprising:
at least one incident imaging device that focuses the incident light beam on at least one portion of the coupler.

15. The monitoring system of claim 14, wherein the at least one imaging device is at a distance from the coupler which is substantially equal to the focal length of the contact lens.

16. The monitoring system of claim 14, further comprising:
at least one output imaging device that focuses the output light beam on the sensor.

17. The monitoring system of claim 1, further comprising:
at least one output imaging device that focuses the output light beam on the sensor.

18. The monitoring system of claim 1, wherein the processing system performs a calibration to remove one or more species from a measured absorption spectrum before calculating the at least one measurable characteristic of the subject.

19. The monitoring system of claim 1, wherein the at least one measurable characteristic of the subject is selected from the group consisting of glucose concentration, blood alcohol level, blood pressure, cholesterol, HDL cholesterol, estrogen, progesterone, and cortisol.

20. The monitoring system of claim 1, wherein the at least one measurable characteristic of the subject is a chemical characteristic of the subject's blood.

21. The monitoring system of claim 1, wherein the at least one measurable characteristic of the subject is a physical characteristic of the subject's blood.

22. The monitoring system of claim 1, wherein the at least one measurable characteristic of the subject is an ocular characteristic.

23. The monitoring system of claim 1, wherein the processing system comprises a user interface.

24. The monitoring system of claim 23, wherein the user interface is selected from the group consisting of a computer screen, and LCD panel, a sound alert, a vibration device, an indicator light, and an LED.

25. A method for monitoring, comprising:
illuminating at least a portion of a subject's eye with a light beam;
coupling the light beam into an aqueous humor of the eye with a coupler contact lens;
outputting the light beam coupled into the aqueous humor with the coupler contact lens;
measuring at least one spectral characteristic of the output light beam; and
calculating one or more measurable characteristics of the subject based on the at least one measured spectral characteristic.

26. The method of claim 25, wherein illuminating at least a portion of the subject's eye with the light beam comprises focusing the light beam on at least a portion of the coupler contact lens with an imaging system.

27. The method of claim 26, further comprising focusing the output light beam onto a sensor prior to measuring the at least one spectral characteristic of the output light beam.

28. The method of claim 26, further comprising, setting the imaging system at a distance from the coupler contact lens which is substantially equal to the effective focal length of the contact lens and a cornea combination.

29. The method of claim 25 further comprising performing a calibration prior to calculating one or more measurable characteristics of the subject based on the at least one measured spectral characteristic.

30. The method of claim 29 wherein performing the calibration comprises removing one or more species from a measured absorption spectrum.

31. The method of claim 25 wherein the at least one measurable characteristic is selected from the group consisting of glucose concentration, blood alcohol level, blood pressure, cholesterol, HDL cholesterol, estrogen, progesterone, and cortisol.

32. A body-worn monitoring system, comprising:
an article which can be worn by a subject;
a light source coupled to the article that illuminates at least a portion of the subject's eye with an incident light beam;
a contact lens with a coupler that:
couples the incident light beam into an aqueous humor of the eye, creating an aqueous light beam; and
couples the aqueous light beam out of the aqueous humor of the eye, creating an output light beam;
a sensor coupled to the article that measures at least one spectral characteristic of the output light beam; and
a processing system coupled to the sensor that calculates at least one measurable characteristic of the subject.

33. The portable body-worn monitoring system of claim 32 wherein the article is selected from the group consisting of eye glasses, sun glasses, hats, helmets, visors, goggles; and masks.

34. The portable body-worn monitoring system of claim 32 wherein the processing system is directly coupled to the sensor.

35. The portable body-worn monitoring system of claim 32, wherein the processing system is remotely coupled to the sensor.

36. A contact lens, comprising:
a first coupler for directing incident light through an aqueous humor;
a second coupler for receiving light directed from the first coupler and directing that light out of the aqueous humor and away from the contact lens.

37. The contact lens of claim 36, wherein the first coupler comprises a diffraction grating, a diffuser, or a reflector.

38. The contact lens of claim 36, wherein the second coupler comprises a diffraction grating, a diffuser, or a reflector.

39. The contact lens of claim 36, wherein the first coupler and the second coupler are continuous.

40. The contact lens of claim 36, further comprising a vision correcting element.

41. A method of manufacturing a contact lens, comprising:
forming a lens substrate; and
forming a coupler on the lens substrate, such that the coupler can direct incident light behind the contact lens, through a medium the contact lens will be worn on, and back out of the contact lens.

42. The method of claim 41, wherein forming the coupler on the lens substrate comprises embossing the lens substrate with an embossing mold.

43. The method of claim 42, wherein the embossing mold comprises a diffraction grating pattern.

44. The method of claim 42, wherein the embossing mold comprises a diffusion pattern.

45. The method of claim 42, wherein the embossing mold comprises a reflective pattern.

46. The method of claim 42, wherein the embossing mold comprises any combination of a diffraction pattern, a diffusion pattern, a reflective pattern, and a refraction pattern.

47. The method of claim 41, wherein forming the coupler on the lens substrate comprises combining two materials with different refractive indexes to form a serrated pattern.

48. The method of claim 41, wherein forming the coupler on the lens substrate comprises adding reflective material at the serrated surface.

49. A contact lens made according to the method of claim 41.
